(19) United States
(12) Patent Application Publication
(10) Pub. No.: US 2025/0107748 A1
(43) Pub. Date: Apr. 3, 2025

(54) SYSTEM AND METHOD OF TRIAGING SEPSIS PATIENTS USING HEART RATE N-VARIABILITY (HRnV)

(71) Applicant: NATIONAL UNIVERSITY OF SINGAPORE, Singapore (SG)

(72) Inventors: Nan LIU, Singapore (SG); Marcus Eng Hock ONG, Singapore (SG)

(73) Assignee: NATIONAL UNIVERSITY OF SINGAPORE, Singapore (SG)

(21) Appl. No.: 18/725,504

(22) PCT Filed: Dec. 19, 2022
(86) PCT No.: PCT/SG2022/050917

§ 371 Date: Jun. 28, 2024

(30) Foreign Application Priority Data
Dec. 28, 2021 (SG) 10202114423W

(51) Int. Cl. A61B 5/00; A61B 5/024; A61B 5/352; G16H 10/60; G16H 50/70

(52) U.S. Cl. CPC A61B 5/412; A61B 5/02405; A61B 5/352; A61B 5/7267; A61B 5/7275; G16H 10/60; G16H 50/70

(57) ABSTRACT

Systems and methods for triaging sepsis patients by receiving a heart rate signal comprising a plurality of heartbeats from a patient, determining a value for each of a plurality of heart rate variability (HRV) parameters and each of a plurality of heart rate n-variability (HRnV) parameters from the heart rate signal and applying the predictive model to the values to determine a sepsis risk category for the patient from a plurality of sepsis risk categories.

### TECHNICAL FIELD

This disclosure generally relates to methods and systems for sepsis risk stratification based on heart activity data.

### BACKGROUND

This background description is provided to generally present the context of the disclosure. Contents of this background section are neither expressly nor impliedly admitted as prior art against the present disclosure.

Sepsis is a life-threatening organ dysfunction arising from a dysregulated host response to infection. Every year, it is estimated to affect more than 50 million people worldwide, resulting in over five million deaths. Prompt recognition of sepsis is beneficial for improving patient outcomes. However, there is no consensus on a method to grade or predict risk of mortality due to sepsis. A wide array of clinical scores has been developed to predict clinical outcomes and assess the severity of illness, but they have severe limitations. A common limitation among these scores is their lack of applicability in the ED or acute settings, as many scores require information (e.g., lab tests) that are time-consuming to generate and often not readily available.

Several common disease severity scoring systems that have been utilized in the ED for the prediction of sepsis mortality, including the Mortality in ED Sepsis (MEDS) score, and intensive care unit (ICU) scoring systems such as the Sequential Organ Failure Assessment (SOFA) score and the Acute Physiology and Chronic Health Evaluation II (APACHE II) score. For example, SOFA score needs laboratory tests such as platelet count and serum creatinine. APACHE II and MEDS scores need more inputs that are difficult to obtain for quick triage.

### SUMMARY

In one embodiment, the present disclosure provides a system for triaging sepsis patients, comprising:
memory;
at least one processor (processor(s)); and
a prediction module comprising a predictive model,
wherein the memory stores instructions that, when executed by the processor(s), cause the processor(s) to:
   receive a heart rate signal comprising a plurality of heart beats from a patient;
   determine a value for each of a plurality of heart rate variability (HRV) parameters and each of a plurality of heart rate n-variability (HRnV) parameters from the heart rate signal; and
   apply the predictive model to the values to determine a sepsis risk category for the patient from a plurality of sepsis risk categories.

In some embodiments, the processor(s) are further configured to receive data, the data comprising at least one of patient demographic data and clinical data, and the processor(s) applies the predictive model to the values to determine a sepsis risk category for the patient by applying the predictive model to the patient data.

In some embodiments, at least one of the HRnV parameters is NN×n, where: N is a number of conventional RR intervals (RRIs) combined to form a single RR n-interval (RRnI), N<<\hat{N} where \hat{N} is a total number of RRIs in the heart rate signal;
1≤n≤N;
x is an absolute variation multiple; and
NNxn is a number of times an absolute difference between successive RRₙIs exceeds xn milliseconds.

In some embodiments, at least one of the HRₙV parameters is pNNxn, where:
N is a number of conventional RR intervals combined to form a single RR n-interval (RRnI), N<<\hat{N} where \hat{N} is a total number of RRIs in the heart rate signal;
1≤n≤N;
x is an absolute variation multiple; and
pNNxn is a number of times an absolute difference between successive
RRₙIs exceeds xn milliseconds, expressed as a proportion of \hat{N}.

Some embodiments relate to a method for triaging sepsis patients, comprising:
receiving a heart rate signal comprising a plurality of heart beats from a patient;
determining a value for each of a plurality of heart rate variability (HRV) parameters and each of a plurality of heart rate n-variability (HRnV) parameters from the heart rate signal; and
applying a predictive model to the values to determine a sepsis risk category for the patient from a plurality of sepsis risk categories.

Some embodiments relate to a system for triaging sepsis patients, comprising:
memory;
at least one processor (processor(s)); and
a prediction module comprising a predictive model,
wherein the memory stores instructions that, when executed by the processor(s), cause the processor(s) to:
   receive electrocardiogram data comprising data of a plurality of heart beats from a patient;
   determine a value for each of a plurality of heart rate variability (HRV) metrics and each of a plurality of heart rate n-variability (HRnV) metrics from the electrocardiogram data;
   transform the heart rate n-variability metrics into respective vector representations;
   determine one or more kernel metrics based on a combination of at least two vector representations;
   apply the predictive model to the values and the kernel metrics to determine a sepsis risk category for the patient from a plurality of sepsis risk categories.

In some embodiments, the kernel metrics are determined using any one of: cosine similarity function, polynomial kernel function, sigmoid kernel function, RBF kernel function, Laplacian kernel function or Chi-squared kernel function.

Some embodiments relate to a method for triaging sepsis patients, comprising:
receiving electrocardiogram data comprising data of a plurality of heart beats from a patient;
determining a value for each of a plurality of heart rate variability (HRV) metrics and each of a plurality of heart rate n-variability (HRnV) metrics from the electrocardiogram data;
transforming the heart rate n-variability metrics into respective vector representations;
determining one or more kernel metrics based on a combination of at least two vector representations;
applying a predictive model to the values and the kernel metrics to determine a sepsis risk category for the patient from a plurality of sepsis risk categories.

### BRIEF DESCRIPTION OF THE DRAWINGS

Some embodiments of systems and methods for sepsis risk categorization, in accordance with the present disclosure, will now be described, by way of non-limiting example only, with reference to the accompanying drawings in which:

FIG. 1 illustrates a flowchart of a method for triaging sepsis patients executable by the system of FIG. 7;

FIG. 2 illustrates a schematic of the RR intervals (RRIs) and the definitions of RRₙI and RRₙIₘ, where 1≤n≤3, 1≤m≤2, parameter m indicates the non-overlapping portion between two successive RRₙIₘ sequences;

FIG. 3 illustrates a flow diagram of an approach to risk-stratify patients of suspected sepsis, where novel HRnV measures and kernelized nonlinear HRV/HRnV features are proposed as components of the prediction model;

FIG. 4 illustrates a flowchart for selection of patients for a study;

FIG. 5 illustrates a chart of ROC curves for HRnV-based prediction models and other benchmark models;

FIG. 6 illustrates a flowchart of a method for triaging sepsis patients incorporating kernel methods; and

FIG. 7 is a block diagram of a system for triaging sepsis patients.

### DETAILED DESCRIPTION

Embodiments relate to systems and methods for processing data obtained from a patent or relating to a patient and determining a sepsis risk category for the patient. The ability to risk-stratify sepsis patients accurately and promptly at the emergency department (ED) is invaluable in informing triage, providing greater confidence to clinical decisions, and guiding management. Embodiments incorporate an artificial intelligence (AI) module and heart rate n-variability (HRnV) metrics to determine a sepsis risk category. HRnV metrics can be calculated from 5-minute electrocardiogram (ECG), to build a fast and accurate triage scoring tool to risk stratify sepsis patients in the ED. Alternatively, HRnV metrics can be calculated from electrocardiogram data of shorter or longer duration depending on the clinical setting.

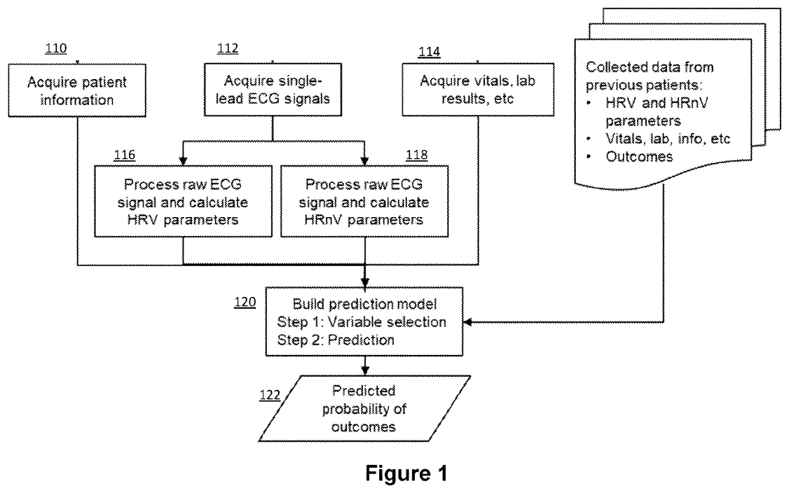

HRV analysis involves measuring the beat-to-beat variation between each R-R interval on an ECG tracing and reflects the autonomic regulation of the cardiovascular system. HRV data is rapidly obtainable on patient presentation even in patients unable to provide a history. FIG. 1 shows an overall architecture of a method of data processing. The artificial intelligence (AI) component includes a prediction module comprising a predictive model. The predictive model of some embodiments included a multivariate predictive model with specific input parameters defined based on a study of data obtained from a clinical setting. The determined sepsis risk category may be numeric categories, for example 1-5 or 1-10 with the number indicating the risk of sepsis for a specific patient. Alternatively, the determined sepsis risk category may be a class label such as low, medium, high, each indicating the risk of sepsis for a specific patient. In some embodiments, the prediction output may be a probability value associated with a probability of a patient outcome.

FIG. 7 illustrates a block diagram of system 730 for triaging sepsis patients. System 730 receives heart rate signal data or ECG data 720 from an ECG device 710. The system 730 comprises at least one processor 732 and memory 734. Memory 734 comprises prediction module program code 736 which is executable by processor 732 to implement a prediction model for determining a sepsis risk category for the patient from a plurality of sepsis risk categories. In addition, memory 734 also comprises program code 742 for HRV, and HRnV computation. System 730 is capable of executing parts of the whole of the methods illustrated in the flowcharts of FIGS. 1, 3 and 6.

### Study Design and Setting

The study involved a retrospective analysis of data from a sample of patients recruited at the Emergency Department of Singapore General Hospital (SGH) between September 2014 to April 2017. SGH is the largest tertiary care hospital in Singapore, with an ED that sees between 300-500 patients daily. Patients are triaged by a trained nurse upon presentation to the ED using the national Singaporean Patient Acuity Category Scale (PACS), a symptom-based triage system without strict physiological criteria. The PACS score ranges from 1 to 4, with a lower score reflecting a greater degree of urgency for consultation with an emergency physician. PACS 1 denotes critically ill patients, PACS 2 patients are non-ambulant, but in a stable condition, PACS 3 patients are ambulant, and patients with PACS 4 are non-emergency cases.

### Study and Population Eligibility

Patients were included in the study if they were 1) aged 21 and above, 2) triaged to either PACS 1 or 2 upon presentation to the ED, 3) clinically suspected to have sepsis by their treating physician, and 4) met at least 2 of 4 Systemic Inflammatory Response Syndrome (SIRS) criteria. The SIRS criteria are temperature (<36° C. or >38° C.), heart rate (>90 beats/min), respiratory rate (>20 breaths/min) and total white blood cell count (<4000/mm³ or >12,000/mm³). Patients were excluded if their ECGs had non-sinus rhythm, a high percentage of noise (artefacts/ectopic beats exceeding 30% of recordings), or if they were on a pacemaker or mechanical ventilator support.

### Data Collection

Five-minute one-lead ECGs were performed on patients who met the inclusion criteria using the X-Series Monitor (ZOLL Medical Corporation, Chelmsford, MA) to obtain heart rate signal/electrocardiogram data. Patient demographics, medical history, laboratory results, and vital signs taken in triage or the ED were retrieved from electronic medical records. The primary outcome of the study was in-hospital mortality (IHM) within 30 days of ED presentation.

### ECG Processing and HRnV Analysis

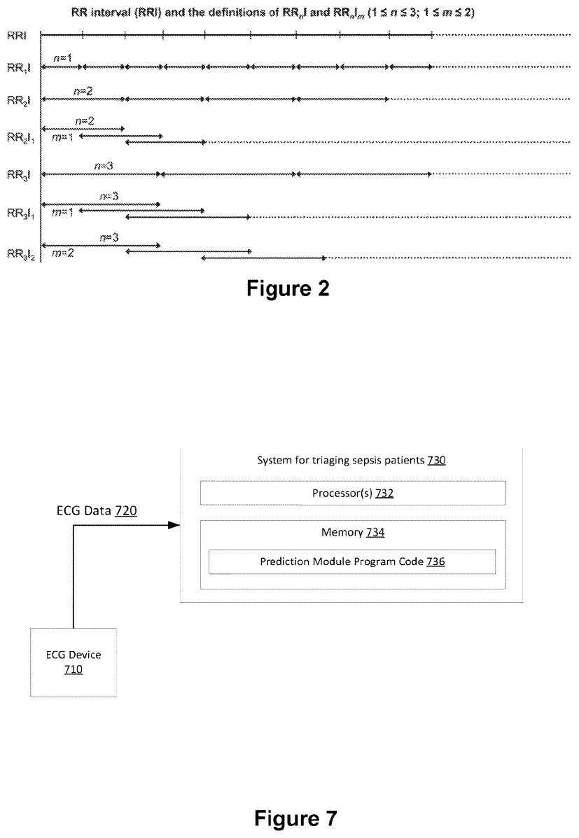

Embodiments comprise processing the ECGs and detected QRS complex to convert the original ECG signals into R-R interval (RRI) sequences (i.e., intervals of consecutive R peaks in ECGs, step 116 of FIG. 1). Some embodiments one or both of two measures to represent beat-to-beat variations in ECG, namely HRnV, and HRₙVₘ (step 118 of FIG. 1). FIG. 2 provides a visual illustration of the HRₙV representation. Conventional HRV analysis evaluates the beat-to-beat variation in an ECG using RR intervals (RRIs). For HRₙV measure, embodiments define a new type of RRI called RRₙI, where 1≤n≤N and N<<\hat{N}. \hat{N} is the total number of RRIs and n is number of conventional RRIs combined to form a single RRₙI. The same quantitative methods of time and frequency domain, and nonlinear analysis were used in conventional HRV analysis can be applied to RRₙI to derive HRₙV parameters. In addition to conventional HRV parameters, HRnV also evaluates two newly created parameters: NN50n and pNN50n. These parameters describe the number of times that the absolute difference between successive RRₙIs exceeds 50×n ms within a given RRnI sequence. pNN50n is the proportion of NN50 intervals divided by the total number of NN (R-R) intervals.

Like HRnV, HRnVm is a measure derived from RRₙIₘ, where 1≤n≤N, 1≤m≤N−1, and N<<\hat{N}. Specifically, m is the number of non-overlapping RRIs for each RRnI. In practice, n can be any number, as can m, though m must be less than n or less than or equal to n. Hence, when m=n, RRₙIₘ becomes RRₙI as there are no overlapping RRIs, resulting in an upper limit of N−1 for m. Utilizing all permissible combinations of n and m, a maximum number of N*(N−1)/2 RRₙIₘ sequences (excluding the RRnI sequence) can be generated from the conventional single RRI sequence. In some embodiments, the upper limit of N=3 is set due to the relatively short ECG samples and the requirement for N<<\hat{N}. Thus, 6 sets of HRₙV parameters were calculated, namely HRV, HR₂V, HR₂V₁, HR₃V, HR₃V₁, and HR₃V₂. In other applications, depending on the length of ECG records, N can set to a different integer value to benefit from longer ECG records.

### Statistical Analysis

Categorical variables were compared between patients who did and did not meet the primary outcome (30-day IHM) using χ² test or Fisher's exact test where appropriate. Continuous variables were checked for normality with the Kolmogorov-Smirnov test. Subsequently, normally distributed variables were presented as mean and standard deviation (SD) and were compared with independent two-tailed t test between groups, while non-normally distributed variables were presented as median and interquartile range (IQR; 25th to 75th percentiles) and compared using the Mann-Whitney U test.

Univariable regression analysis was conducted on traditional HRV parameters, novel HRnV parameters and demographic and clinical variables. Each variable was evaluated as an individual predictor of the primary outcome (30-day IHM) using binary logistic regression with odds ratio (OR), 95% confidence interval (CI), and p-value reported. For multivariable regression analysis, the study adjusted for age, temperature, systolic blood pressure, heart rate, and Glasgow Coma Scale (GCS) as these variables were either shown to be significant predictors of sepsis mortality in previous literature or are included in well-established sepsis scoring systems such as the National Early Warning Score (NEWS), Modified Early Warning Score (MEWS), qSOFA, or APACHE II. HRV and HRnV parameters were included in the multivariable analysis if they achieved p<0.2 in the univariable analysis. Included variables were then checked for collinearity using Pearson's R correlation. For each collinear pair, the variable with the higher p-value on univariate analysis was eliminated until no collinear pairs remained.

The remaining variables were then fed into a backward stepwise multivariable logistic regression model, which used p<0.1 as an endpoint. Embodiments defined statistical significance at p<0.05. Backward elimination was chosen for the stepwise variable selection because it has the advantage to assess the joint predictive ability of variables, and it removes the least essential variables. The eliminated variables were not allowed to re-enter the model in some embodiments. In other embodiments, every combination of variables was examined. Examination of every combination of variables required significant computing resources with the risk of overfitting the model when the number of variables is large.

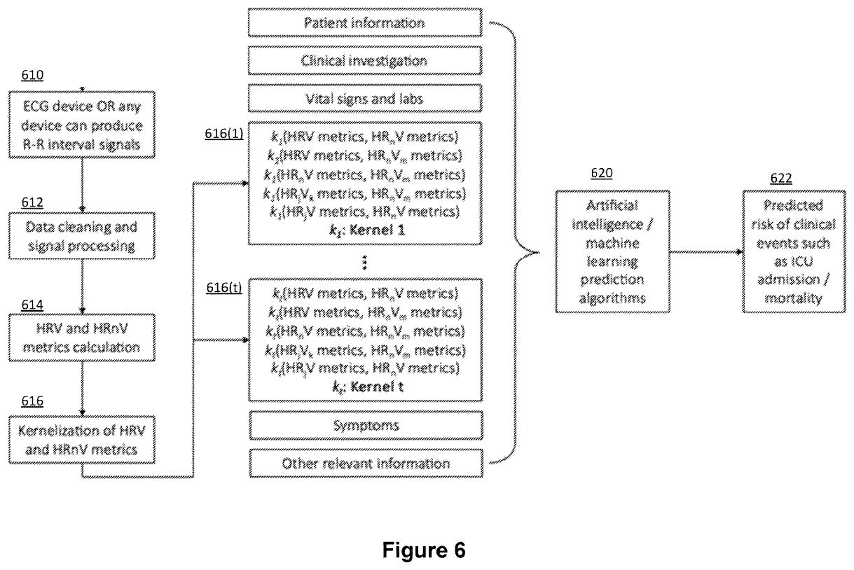

In predictive modelling with the selected variables, the embodiments conducted 10-fold cross-validation to avoid overfitting in the evaluating/prediction models. The entire dataset was split into 10 non-overlapping subsets of equivalent size and then used nine subsets to build a model and validated the model with the remaining one subset. Embodiments repeated the above process ten times to ensure that each of the ten subsets could be validated. Subsequently, a receiver operating characteristic (ROC) curve was plotted to assess the predictive ability of the multivariable regression model and compared against other established disease scoring systems on their area under the curve (AUC) as illustrated in FIG. 6.

Missing data were addressed by median imputation, in consideration of the low proportion of missing data (<0.3%) for each variable, the nature of variables, and recommendations for missing data in clinical trials. All statistical analyses were carried out using Python version 3.8.0 (Python Software Foundation, Delaware, USA) using the SciPy library (version 1.3.1). Regression models were built using the StatsModels library (version 0.10.2) and scikit-learn library (version 0.22). Table 1 below provides a summary of the statistical analysis of the data in the study.

TABLE 1
List of HRV (traditional) and HRnV (novel) parameters

| HRV | HR₂V | HR₂V₁ | HR₃V | HR₃V₁ | HR₃V₂ |
|---|---|---|---|---|---|
| Mean NN (s) | HR₂V Mean NN | HR₂V₁ Mean NN | HR₃V Mean NN | HR₃V₁ Mean NN | HR₃V₂ Mean NN |
| SDNN (s) | HR₂V SDNN | HR₂V₁ SDNN | HR₃V SDNN | HR₃V₁ SDNN | HR₃V₂ SDNN |
| RMSSD (s) | HR₂V RMSSD | HR₂V₁ RMSSD | HR₃V RMSSD | HR₃V₁ RMSSD | HR₃V₂ RMSSD |
| Skewness | HR₂V Skewness | HR₂V₁ Skewness | HR₃V Skewness | HR₃V₁ Skewness | HR₃V₂ Skewness |
| Kurtosis | HR₂V Kurtosis | HR₂V₁ Kurtosis | HR₃V Kurtosis | HR₃V₁ Kurtosis | HR₃V₂ Kurtosis |
| Triangular | HR₂V Triangular | HR₂V₁ Triangular | HR₃V Triangular | HR₃V₁ Triangular | HR₃V₂ Triangular |
| index | index | index | index | index | index |
| NN50 (count) | HR₂V NN50 | HR₂V₁ NN50 | HR₃V NN50 | HR₃V₁ NN50 | HR₃V₂ NN50 |
| pNN50 (%) | HR₂V pNN50 | HR₂V₁ pNN50 | HR₃V pNN50 | HR₃V₁ pNN50 | HR₃V₂ pNN50 |
| — | HR₂V NN50n | HR₂V₁ NN50n | HR₃V NN50n | HR₃V₁ NN50n | HR₃V₂ NN50n |
| — | HR₂V pNN50n | HR₂V₁ pNN50n | HR₃V pNN50n | HR₃V₁ pNN50n | HR₃V₂ pNN50n |
| Total power (ms²) | HR₂V Total power | HR₂V₁ Total power | HR₃V Total power | HR₃V₁ Total power | HR₃V₂ Total power |
| VLF power (ms²) | HR₂V VLF power | HR₂V₁ VLF power | HR₃V VLF power | HR₃V₁ VLF power | HR₃V₂ VLF power |
| LF power (ms²) | HR₂V LF power | HR₂V₁ LF power | HR₃V LF power | HR₃V₁ LF power | HR₃V₂ LF power |
| HF power (ms²) | HR₂V HF power | HR₂V₁ HF power | HR₃V HF power | HR₃V₁ HF power | HR₃V₂ HF power |
| LF power norm | HR₂V LF power | HR₂V₁ LF power | HR₃V LF power | HR₃V₁ LF power | HR₃V₂ LF power |
| (nu) | norm | norm | norm | norm | norm |
| HF power norm | HR₂V HF power | HR₂V₁ HF power | HR₃V HF power | HR₃V₁ HF power | HR₃V₂ HF power |
| (nu) | norm | norm | nom | nom | norm |
| LF/HF | HR₂V LF/HF | HR₂V₁ LF/HF | HR₃V LF/HF | HR₃V₁ LF/HF | HR₃V₂ LF/HF |
| Poincaré SD1 | HR₂V Poincaré | HR₂V₁ Poincaré | HR₃V Poincaré | HR₃V₁ Poincaré | HR₃V₂ Poincaré |
| (ms) | SD1 | SD1 | SD1 | SD1 | SD1 |
| Poincaré SD2 | HR₂V Poincaré | HR₂V₁ Poincaré | HR₃VPoincaré | HR₃V₁ Poincaré | HR₃V₂ Poincaré |
| (ms) | SD2 | SD2 | SD2 | SD2 | SD2 |
| Poincaré | HR₂V Poincaré | HR₂V₁ Poincaré | HR₃V Poincaré | HR₃V₁ Poincaré | HR₃V₂ Poincaré |
| SD1/SD2 ratio | SD1/SD2 | SD1/SD2 | SD1/SD2 | SD1/SD2 | SD1/SD2 |
| SampEn | HR₂V SampEn | HR₂V₁ SampEn | HR₃V SampEn | HR₃V₁ SampEn | HR₃V₂ SampEn |
| ApEn | HR₂V ApEn | HR₂V₁ ApEn | HR₃V ApEn | HR₃V₁ ApEn | HR₃V₂ ApEn |
| DFA, α1 | HR₂V DFA, α1 | HR₂V₁ DFA, α1 | HR₃V DFA, α1 | HR₃V₁ DFA, α1 | HR₃V₂ DFA, α1 |
| DFA, α2 | HR₂V DFA, α2 | HR₂V₁ DFA, α2 | HR₃V DFA, α2 | HR₃V₁ DFA, α2 | HR₃V₂ DFA, α2 |

### Patient Recruitment

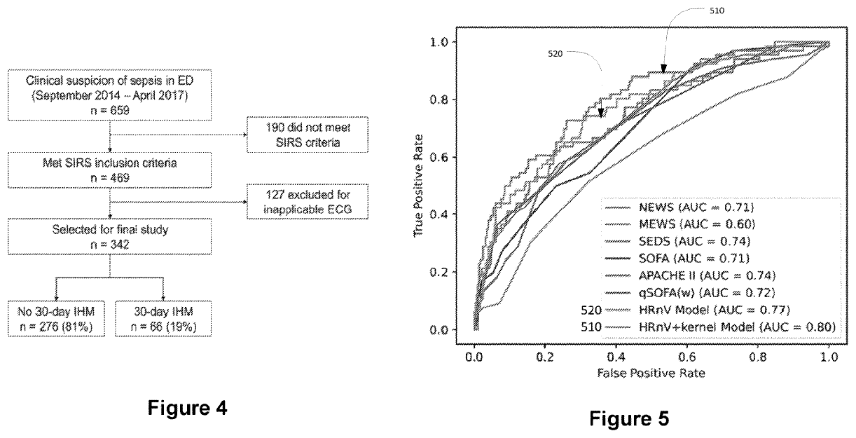

FIG. 4 presents the patient recruitment flowchart of the study of some embodiments. Of the 659 patients that were initially recruited, 190 patients did not meet the SIRS criteria, and 127 patients had inapplicable ECG readings. Three hundred forty-two patients were included for analysis and classified depending on whether they met the primary outcome of 30-day IHM (n=66, 19%) or did not meet the primary outcome (n=276, 81%).

### Baseline Characteristics and Clinical Parameters

Table 2 illustrates baseline characteristics and clinical parameters of patients who met and did not meet with 30-day IHM. Patients who met with 30-day IHM were older and presented with higher respiratory rates but lower temperatures, systolic blood pressures (SBP) and GCS scores, compared to patients who did not meet with 30-day IHM. The worst recorded values of respiratory rate, GCS, and SBP during each patient's ED stay were also significantly more abnormal in patients that met with 30-day IHM. The difference in disposition from the ED was significant, with a larger proportion of patients who eventually met with 30-day IHM requiring admission to the ICU as compared to patients who did not meet with 30-day IHM (16.7% vs 4.3%, p=0.001). Additionally, a larger proportion of patients who met with 30-day IHM had a respiratory source of infection (45.5% vs 27.2%, p=0.006) while a smaller proportion had a source of infection originating from the urinary tract (7.6% vs 25.7%, p=0.003) when compared to patients who did not meet with 30-day IHM. No significant differences were detected in gender, PACS status, ethnicity, or medical history between both groups.

TABLE 2
Baseline Characteristics and Clinical Parameters

| | No 30-day | 30-day | |
|---|---|---|---|
| Variable | IHM (n = 276) | IHM (n = 66) | P-value |
| age, mean (SD) | 65.8 (16.1) | 73. [symbol] (14.8) | 0.001 [symbol] |
| Male gender, [symbol] (%) | 1 [symbol] 4.0 (52.2) | 30. [symbol] (45. [symbol] ) | 0.399 |
| [symbol] to high [symbol] (%) | 254.0 (92.0) | 64.0 ( [symbol] ) | 0.19 |
| [symbol] criteria [symbol] (%) | 25 [symbol] .0 (90.6) | 58.0 (87.9) | 0. [symbol] 7 |
| Race, n (%) | | | 0.946 |
| Chinese | 20 [symbol] (73.6) | [symbol] 9.0 (74.2) | 0.967 |
| [symbol] | [symbol] 0.0 ( [symbol] 4.5) | 8.0 (12.1) | 0.763 |
| Indian | 2 [symbol] .0 (7.6) | 6.0 (9.1) | 0.883 |
| Other | 12.0 (4.3) | 3.0 (4.5) | 1 |
| Disposition from ED, n (%) | | | 0. [symbol] |
| Intensive care unit | 12.0 (4.3) | 11. [symbol] (16.7) | 0. [symbol] |
| high dependency unit | 28.0 (10.1) | 3.0 (4.5) | 0.231 |
| General ward | 236.0 (85.5) | 52.0 (78.8) | 0.247 |
| Medical history, n (%) | | | |
| Ischemic heart disease | 73.0 (26.4) | 21.0 (31.8) | 0.4 [symbol] 9 |
| Diabetes | [symbol] .0 (40.2) | 24.0 (36.4) | 0.663 |
| Hypertension | 156.0 ( [symbol] 6.5) | 35.0 (53.0) | 0.708 |
| Cancer | 79.0 (28.6) | 23.0 (34.8) | 0.399 |
| Serious Infections | 117.0 (4 [symbol] .4) | 28.6 (42.4) | 0.894 |
| Source of infection, n (%) | | | |
| Respiratory | 73.0 (27.2) | 30.0 (45.5) | 0.0 [symbol] |
| Urinary tract | 71.0 (25.7) | 5.0 (7.6) | 0.0 [symbol] |
| Gastrointestinal | 18.0 (6.5) | 4.0 (6.3) | 1 |
| Musculoskeletal | 11.0 (4.0) | 3.0 (4.5) | 0.738 |
| [symbol] | 20.0 (7.2) | 0.0 (0.0) | 0.018 [symbol] |
| Peritoneum | 3.0 (1.1) | 2.0 ( [symbol] .0) | 0.248 |
| Ski [symbol] | 3.0 (1.1) | 0.0 (0.0) | 1 |
| Li [symbol] | 7.0 (2.5) | 3.0 (0.0) | 0.354 |
| Cardiac | 7.0 (2.5) | 2.0 (3.0) | 0.6 [symbol] |
| Central nervous system | 1.0 (0.4) | 0.0 (0.0) | 1 |
| Unknown | 23.0 (8.3) | 12.0 (18.2) | 0.032 [symbol] |
| No infection | 37.0 (13.4) | 8.0 (12.1) | 0. [symbol] |
| Vital sign predictors, | | | |
| mean (SD) or median IQR) | | | |
| Heart rate, bpm | 114.2 (23.3) | 122.7 (26.0) | 0.652 |
| White blood cell count | 14.0 (7.7) | 13.0 (9.6) | 0.36 |
| Diastolic BP, mmHg | 63.0 (19.5) | 59.7 (17.4) | 0.213 |
| Temperatue, ° C. | 38.1 (37.1-38.8) | 37.2 (36. [symbol] -3 [symbol] .0) | <001 [symbol] |
| Respiratory rate, bpm | 19.0 (18.0-21.0) | 22.0 (19.8-25.0) | <001 [symbol] |
| Respiratory rate (worst), bpm | 21.0 (19,8-24.0) | 26.0 ( [symbol] 2.0-30.0) | <001 [symbol] |
| Systolic BP, mmHg | [symbol] (86.0-139.0) | [symbol] (78.0-118.5) | 0.0 [symbol] |
| Systolic BP (worst), mmHg | 90.0 (77.9-109.2) | 78.0 (63. [symbol] -94.8) | <001 [symbol] |
| [symbol] (3-15) | 33.8 (3.0) | 11.7 (4.1) | 0.00 [symbol] |
| W [symbol] (3-1.5) | 33.3 (3.1) | 11.7 (4.1) | 0.002 [symbol] |

[symbol]

p < 0.05,
IHM: In hospital mortality;
SD: Standard deviation;
IQR: Interquartile range;
BP: Blood pressure;
GCS: Glasgow Coma Scale;
ED: Emergency department. [symbol]
indicates data missing or illegible when filed

### HRV and HRnV Parameter Description and Univariate Analysis

Table 3 presents the descriptive analysis of HRV and HRₙV parameters. In the study, N was set as 3 and HR₂V, HR₂V₁, HR₃V, HR₃V₁ and HR₃V₂ parameters/metrics were calculated. Among time domain parameters such as mean NN and SDNN, HRₙV and HRₙVₘ values are generally directly proportional to n and increase when n increases. HR₂V SampEn and HR3V SampEn were considerably larger than SampEn parameters of HRV, HR2V1, HR3V1, and HR3V2. This was because of insufficient data points since our ECG recordings were only five minutes long. HR₂V₁, HR₃V₁ and HR₃V₂ did not encounter this limitation as more data points were available from a calculation using overlapping RRₙIₘ sequences.

Table 4 shows the results of univariable analysis of HRV and HRₙV parameters. Of 142 HRV and HRₙV parameters, 85 were significantly different between the two outcome groups. Specifically, 14 HRV, 14 HR₂V, 16 HR₂V₁, 11 HR₃V, 16 HR₃V₁, and 14 HR3V2 parameters were statistically significant. In at least four out of six HRnV measures, RMSSD, kurtosis, NN50, pNN50, NN50n, pNN50n, HF power, HF power norm, Poincare SD1, and Poincare SD1/SD2 were significantly higher, while LF power norm and DFA α2 were significantly lower in patients who met the primary outcome compared to those who did not. Additionally, VLF power and DFA α1 were not significant in HRV analysis but were statistically significant in several HRnV measures.

Overall, six baseline characteristics (age and vital signs at triage including temperature, respiratory rate, SpO₂, SBP and GCS), 17 HRV parameters, and 96 HRnV parameters had p<0.2 on univariable analysis. After collinearity assessment, the remaining 87 variables were entered into a stepwise-selection regression model.

TABLE 3
Descriptive analyses of heart rate variability (HRV)
and heart rate n-variability HRnV) parameters.

| | HRV | HR₂V | HR₃V [symbol] | HR₃V | HR₃V [symbol] | HR₃V₂ |
|---|---|---|---|---|---|---|
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | — | [symbol] | [symbol] | [symbol] | [symbol] | [symbol] |
| [symbol] | — | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

[symbol]

indicates data missing or illegible when filed

TABLE 4
Univariable analysis of HRV and HRnV parameters

| | HRV | | HR₃V | | HR₃V | |
|---|---|---|---|---|---|---|
| | OR (95%Cl) | p | OR (95%Cl) | p | OR (95%Cl) | p |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | — | [symbol] | [symbol] | [symbol] | [symbol] | [symbol] |
| [symbol] | — | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| | HR₂V [symbol] | | HR [symbol] V [symbol] | | HR [symbol] V₃ | |
| | OR (95%Cl) | p | OR (95%Cl) | p | OR (95%Cl) | p |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |
| [symbol] | [symbol] | [symbol] | [symbol] | [symbol] | [symbol] | [symbol] |

[symbol]

[symbol]

indicates data missing or illegible when filed

TABLE 5
Multivariable analysis of HRV and HRnV
parameters on 30-day in-hospital mortality

| | Adjusted Odds | |
|---|---|---|
| Variables | Ratio (95% CD) | p-value |
| Vital signs | | |
| Respiratory rate | 1.168 (1.085 to 1.257) | <0.001 [symbol] |
| Systolic blood pressure | 0.978 (0.966 to 0.990) | 0.001 [symbol] |
| Glasgow coma scale | 0.843 (0.769 to 0.929) | 0.001 [symbol] |
| SpO₂ | 0.892 (0.838 to 0.950) | <0.001 [symbol] |
| HRV parameters | | |
| HRV total power | 1.000 (1.000 to 1.001) | 0.019 [symbol] |
| HRV Poincare SD [symbol] | 0.948 (0.893 to 1.007) | 0.081 |
| HRnV parameters | | |
| HR [symbol] V NN5O | 1.270 (0.967 to 1.667) | 0.085 |
| HR [symbol] V HFE power | 1.002 (1:000 to 1.003) | 0.076 |
| HR [symbol] V pNN50 | 0.290 (0.115 to 0.732) | 0.009 [symbol] |
| HR [symbol] V ApEn | 0.106 (0.013 to 0.877) | 0.037 [symbol] |
| HR₂V₁ NN50 | 0.808 (0.682 to 0.958) | 0.014 [symbol] |
| HR₃V₁ pNN50 | 5.700 (1.784 to 18.213) | 0.003 [symbol] |
| HR₃V₁ LF/HF | 0.251 (0.07) to 0.880) | 0.031 [symbol] |
| HR₃V₁ pNN50 | 1.229 (0.988 to 1.528) | 0.064 |
| HR₃V₁ NN50 | 1.098 (0.999 to 1.206) | 0.053 |
| HR₃V₁ LF/HF | 2.241 (1.252 to 4.013) | 0.007 |
| HR₃V₁ pNN50 | 0.473 (0.247 to 0.906) | 0.024 [symbol] |
| HR₃V₁ NN50n | 0.794 (0.664 to 0.950) | 0.012 [symbol] |
| HR₃V₁ HF power norm | 1.093 (1.044 to 1.144) | <0.001 [symbol] |
| HR₃V₁ DFA α1 | 14.189 (1.009 to 199.510) | 0.049 [symbol] |
| HR₃V₂ NN50n | 1.713 (1.202 (0 2.443) | 0.003 [symbol] |

[symbol]

p < 0.05.
HRV: Heart rate variability;
CI: Confidence interval;
NN50: The number of times that the absolute difference between two successive R-R intervals exceeds 50 ms;
pNN50: NN50 divided by the total number of R-R-intervals;
NN50n: The number of times that the absolute difference between 2 successive RR [symbol] I/RR [symbol] I [symbol] sequences exceeds 50 [symbol] n ms;
LF: Low frequency;
HP: High frequency,
SD: Standard deviation;
ApEn: Approximate entropy
DFA: Detrended fluctuation analys [symbol]
indicates data missing or illegible when filed

### Multivariable Analysis and ROC Analysis

Table 5 presents the multivariable analysis of variables found to be significantly different on univariable analysis. A total of 21 out of 87 variables were selected through stepwise selection. Of the 21 variables, 16 showed p<0.05. These include vital signs such as respiratory rate (OR=1.168; 95% CI 1.085-1.257; p<0.001), SBP (OR=0.978; 95% CI 0.966-0.990; p=0.001), SpO₂ (OR=0.892; 95% CI 0.838-0.950; p=<0.001), and GCS (OR=0.845; 95% CI 0.769-0.929; p=0.001), and HRₙV measures such as HR₂V₁ NN50 (OR=0.808; 95% CI 0.682-0.958; p=0.014), HR₂V pNN50 (OR=0.290; 95% CI 0.115-0.732; p=0.009), HR₂V₁ pNN50 (OR=5.700; 95% CI 1.784-18.213; p=0.003), HR₂V ApEn (OR=0.106; 95% CI 0.013-0.877; p=0.037) and several HR₃V₁ and HR₃V₂ parameters which demonstrated strong predictive power in assessing the risk for 30-day IHM. The final multivariable predictive model consisted of four vital signs, two traditional HRV parameters, and 15 novel HRₙV parameters. Hereafter, we refer to this model as the HRₙV model.

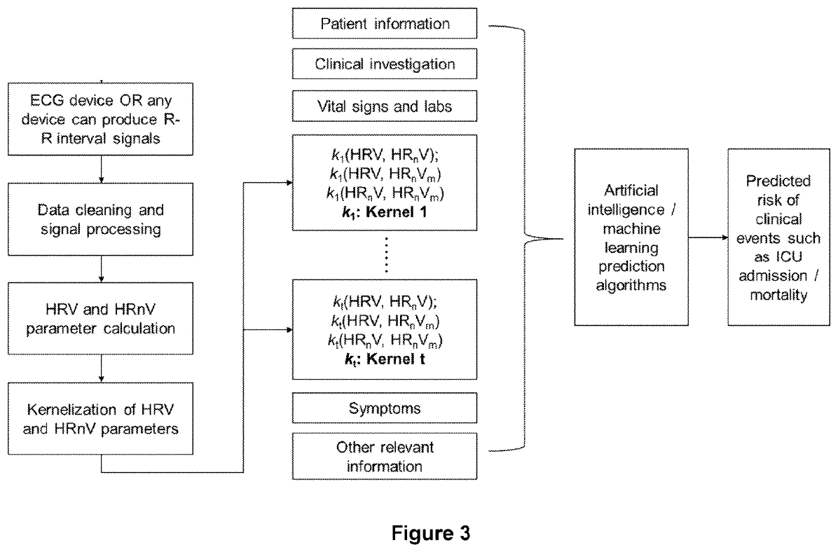

ROC curves were plotted for assessment of the HRₙV model and compared against established disease severity scoring systems to predict 30-day IHM in patients presenting to the ED with sepsis (FIG. 3). The AUC of the HRₙV model based on 10-fold cross-validation was 0.77 (95% CI: 0.70-0.84), outperforming the AUC of NEWS 0.71 (95% CI: 0.64-0.78), MEWS 0.60 (95% CI: 0.53-0.67), SOFA 0.71 (95% CI: 0.64-0.78), APACHE II 0.74 (95% CI: 0.68-0.80), and the patient's worst qSOFA value 0.72 (95% CI: 0.65-0.79).

### Variables for Predictive Modelling

The inputs to the AI scoring tool (prediction model) include the above-mentioned patient information (step 110 of FIG. 1) and results of clinical investigations (step 114 of FIG. 1), as well as HRV and HRnV parameters. In some embodiments, a subset of the parameters/metrics described above may be provided as input to the prediction model. The scoring tool may use one or more than of the following HRV and HRnV parameters: Mean NN(s), SDNN(s), RMSSD(s), Skewness, Kurtosis, Triangular index, NN50 (count), pNN50 (%), Total power (ms2), VLF power (ms2), LF power (ms2), HF power (ms2), LF power norm (nu), HF power norm (nu), LF/HF, Poincaré SD1 (ms), Poincaré SD2 (ms), Poincaré SD1/SD2 ratio, Sample entropy, Approximate entropy, DFA α1, and DFA α2. The tool may also use at least one of HRnV and HRnVm parameters.

### Predictive Modeling With Machine Learning Algorithm

At step 120 of FIG. 1, the embodiments build a prediction model based on the data obtained in the study. This comprises the selection of relevant variables for input to the model, and the actual step of prediction by inputting variable values/parameters associated with a specific patient to obtain the predicted outcome 122. In the study, the inputs to the prediction models were all or a subset of the 22 HRV parameters, 120 HRnV parameters (illustrated in Table 1) and several baseline characteristics such as patient demographics, vital signs and lab test results. Each variable was evaluated as a predictor of the primary outcome (i.e., 30-day IHM). In multivariable modelling analysis, embodiments included age, systolic blood pressure, heart rate, temperature, and Glasgow Coma Scale (GCS) score as these variables either have shown to be significant predictors of sepsis mortality or are included in established sepsis scoring systems such as NEWS28, MEWS29, MEDS, qSOFA, or APACHE II. Embodiments also performed evaluation for variable collinearity using an R2 correlation coefficient.

The study evaluated the predictive value of novel HRnV measures (HRnV and HRnVm) in estimating the risk of 30-day IHM in patients presenting to the ED with sepsis. In addition to the 22 traditional HRV parameters, the study derived an additional 120 HRnV parameters, 71 of which were found statistically significant in their association with the primary outcome. The newly generated HRnV parameters augment the number of candidate predictors and have demonstrated improved predictive ability for sepsis mortality.

In HRnV measures, the parameters, NN50n and pNN50n, were significantly associated with mortality in the univariate analysis. They characterize the number of times that the absolute difference between two successive RRnI sequences exceeds 50×n ms, by assuming the absolute difference may be magnified when the corresponding RRnI is n times longer than RRI. The composite HRnV model derived from multivariable logistic regression achieved the highest AUC on ROC analysis and outperformed other established disease scoring systems such as NEWS, MEWS, SOFA, and APACHE II for the prediction of 30-day IHM in patients presenting to the ED with sepsis.

In addition to demonstrating the improved predictive ability for sepsis mortality, the HRnV model is made even more relevant in its capacity for rapid and objective prognostication where only vital signs and parameters calculated from five-minute ECG tracings are needed. Many established disease severity scores require invasive tests, which need a long turnaround time and resources to obtain or include subjective parameters that involve interrater variability while scoring. Among disease severity scores, the MEDS score, explicitly developed for risk stratification of septic patients in the ED, suffers from some of these limitations and its adoption has thus not been widespread. Consequently, MEDS was not included in our comparison. APACHE II and SOFA scores initially designed for use in the intensive care unit (ICU) setting similarly require invasive investigations to calculate its score. In these aspects, the HRnV model which is derived from vital signs taken on ED presentation, and HRV and HRnV parameters calculated from five-minute ECG tracings, may overcome these limitations and provide a rapid, objective, and accurate risk assessment of the septic patient. A triage system with these characteristics is invaluable to the physician and can aid in risk stratification, clinical management, patient disposition, and accurate patient classification for administrative or research purposes. Furthermore, our HRnV analysis and modelling modules can be readily integrated into a monitoring device, making the real-time prediction of sepsis severity a feasible task.

In some embodiments, in addition to the heart rate signal data, the prediction model may also generate a sepsis risk category for a patient based on at least one of the patient demographic data and clinical data.

### Kernel Methods/Functions for HRnV Analysis

Some embodiments apply kernel methods to HRV and HRnV metrics to capture the nonlinear interactions introduced by HRnV and to improve the accuracy of the prediction model without the need of acquiring additional measurements from patients.

Some embodiments adopt kernel functions (e.g. polynomial kernel and radial basis function kernel) to combine HRV and HRnV parameters in a nonlinear manner. The rationale for this algorithm is that some HRV and HRnV parameters are linearly linked, which could reduce the discriminatory power in predicting adverse clinical outcomes. With HRV, HRₙV, and HRₙVₘ as inputs, we can create various types of kernel function-mapped new nonlinear feature vectors, i.e. k(HRV, HRnV), k(HRV, HRnVm), k(HRnV, HRnVm) where k(⋅) is the kernel function and HRV, HRnV and HRnVm are vectors of identical dimensions. Multiple kernel functions may be used such that multiple sets of converted feature vectors are available. When choosing kₜ as the kernel function, the parameters/metrics may include kₜ(HRV, HRnV), kₜ(HRV, HRnVm), and kₜ(HRnV, HRnVm), where “t” is an integer of any value depending on the choice of kernel functions. Newly created kernel features may be integrated with basic patient information and clinical investigation results as inputs to feed into a prediction algorithm, such as neural network, support vector machine, decision tree, random forest, boosting, or ensemble learner etc.

Since each HRV or HRnV analysis is a conventional HRV analysis performed on the corresponding interval sequence (i.e., RRI or RRₙIₘ), some embodiments group the metrics from each HRV or HRnV analysis as a metrics vector. For a single patient, HRV and multiple HRnV analyses can be performed using the same source RRI measured from the patient, resulting a number of metrics vectors (vector representations) of the same length (e.g., HRV metrics vector, HR₃V₂ metrics vector). Kernel methods can then be applied on these metrics vectors to reflect the nonlinear interactions between them.

Considering two different HRnV metrics vectors obtained from the same patient, V_{HR}_{ⱼ}_{V}_{ₖ} and V_{HR}_{ₙ}_{V}_{ₘ} of the same length. Kernel methods involve calculating the inner product of the two vectors in a high dimensional vector space, measuring the nonlinear interactions between the vectors in their original space. The inner product I_{jk×nm}^{k}^{ₜ} from kernel kₜ is given by:

I_{jk×nm}^{k}^{ₜ}=kₜ(V_{HR}_{ⱼ}_{V}_{ₖ},V_{HR}_{ₙ}_{V}_{ₘ})

Some embodiments use one or more than one of six different forms of kernel for kₜ. The six different forms include cosine similarity, polynomial kernel, sigmoid kernel, RBF kernel, Laplacian kernel, and Chi-squared kernel. Table 6 provides the detail formula for each of the kernel methods. Other suitable forms of kernel functions or methods may be employed by the embodiments.

TABLE 6

| | Formula for kernel methods |
|---|---|
| Cosine similarity | $k ( x , y ) = \frac{{x}^{⊤} y}{‖ x ‖ ‖ y ‖} , where ‖ \cdot ‖ represents the Euclidean ( L 2 ) norm .$ |
| Polynomial | k(x, y) = (γ * x^{T}y + c₀)^{d}, where d represents the kernel degree. γ and c₀ are kernel param- |
| kernel | eters. |
| Sigmoid | k(x, y) = tanh(γ * x^{T}y + c₀), where γ and c₀ are kernel parameters. |
| kernel | |
| RBF kernel | k(x, y) = exp(−γ * \|\|x − y\|\|₂), where \|\|·\|\|₂ represents the L2 norm. γ is the kernel parameter. |
| Laplacian | k(x, y) = exp(−γ * \|\|x − y\|\|₁), where \|\|·\|\|₁ represents the L1 norm. γ is the kernel parameter. |
| kernel | |
| Chi-squared kernel | $k ( x , y ) = exp ( - γ * {\sum }_{i} \frac{{( x [ i ] - y [ i ] )}^{2}}{x [ i ] + y [ i ]} ) , where y is the kernel parameter .$ |

The inner products obtained from these kernel methods can then be treated as candidates (inputs/parameters) for the stepwise model or the prediction model.

In some embodiments, HRnV analyses were performed on the source RRI from each of the patients with both HRnV parameters n and m up to 3, resulting in 6 different HRnV metrics vectors (including the conventional HRV metrics vector). The nonlinear interactions were measured between every unique pair from the 6 metrics vectors, providing 15 inner products for each of the kernel methods. In total, 90 different inner products were added to each patient's data.

FIG. 6 provides a graphical illustration of various steps of kernel methods. At step 610, R-R interval signals (electrocardiogram data) is obtained by system 730. The received data is subjected to cleaning and signal processing operations at step 612. Step 614, HRV and HRₙV metrics are computed by execution of program code 742. At step 616, the HRV and HRₙV metrics data is transformed into vectors and subjected to kernelization using any one or more than one methods listed in table X? above. Examples of kernel metrics are illustrated in blocks 616(1) and 616(t). One or more kernel metrics may be computed based on various potential combinations of HRV and HRₙV metrics. The computed kernel metrics are provided as input to the prediction module at step 620 to determine a sepsis risk category for the patient from a plurality of sepsis risk categories.

### Results

A prediction model incorporating kernel methods was applied to the patient data from SGH. One difference between the proposed model and our previous model is the addition of 90 inner products from 6 different kernel methods. The ROC curves with AUCs of all models obtained from 10-fold cross validation is shown in FIG. 5. As illustrated in FIG. 5, a prediction model incorporating the kernel methods resulted in an AUC of 0.8 (graph 510). A prediction model according to the embodiments not incorporating the kernel method resulted in an AUC of 0.77 (graph 520).

The embodiments enable patient triage and quick risk stratification in the EDs and clinics. Since the core technique is ECG-derived HRnV, it is possible to extend the application of the embodiments to any areas where single-lead ECGs are available. This could be in healthcare institutions or at home or in an ambulance. Other than medical devices, it is possible to incorporate the embodiments in home-based wearable ECG/heart rate monitoring devices etc.

The use of novel HRV measures (HRnV and HRnVm) provides additional power to predictive models in the risk stratification of patients who present to the ED with potential sepsis. When included in a model with other clinical variables, the HRnV model outperforms traditional risk stratification scoring systems as shown in our preliminary results. Prospective multi-cen-tre cohort studies would be valuable in validating the effectiveness of the HRnV parameters. The use of HRnV may allow for a rapid, objective, and accurate means of patient risk stratification for sepsis severity and mortality.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, features, functions, operations, or steps, any of these embodiments may include any combination or permutation of any of the components, elements, features, functions, operations, or steps described or illustrated anywhere herein that a person having ordinary skill in the art would comprehend. Although this disclosure describes or illustrates particular embodiments as providing particular advantages, particular embodiments may provide none, some, or all of these advantages.

1-21. (canceled)

22. A system for triaging sepsis patients, comprising:
memory;
at least one processor (processor(s)); and
a prediction module comprising a predictive model,
wherein the memory stores instructions that, when executed by the processor(s), cause the processor(s) to:
receive electrocardiogram data (ECG data) comprising data of a plurality of heart beats from a patient;
determine a value for each of a plurality of heart rate variability (HRV) metrics and each of a plurality of heart rate n-variability (HRnV) metrics from the ECG data;
transform the heart rate n-variability metrics into respective vector representations;
determine one or more kernel metrics based on a combination of at least two vector representations;
apply the predictive model to the values and the kernel metrics to determine a sepsis risk category for the patient from a plurality of sepsis risk categories.

23. The system of claim 22, wherein the kernel metrics are determined using any one of: cosine similarity function, polynomial kernel function, sigmoid kernel function, RBF kernel function, Laplacian kernel function or Chi-squared kernel function.

24. The system of claim 22, wherein the processor(s) are further configured to receive data, the data comprising at least one of patient demographic data and clinical data, and the processor(s) applies the predictive model to the values to determine a sepsis risk category for the patient by applying the predictive model to the patient data.

25. The system of claim 22, wherein at least one of the HRnV parameters is NNxn, where: N is a number of conventional RR intervals (RRIs) combined to form a single RR n-interval (RRnI), N<<\hat{N} where \hat{N} is a total number of RRIs in the ECG data;
1≤n≤N;
x is an absolute variation multiple; and
NNxn is a number of times an absolute difference between successive RRₙIs exceeds xn milliseconds.

26. The system of claim 22, wherein at least one of the HRₙV parameters is pNNxn, where:
N is a number of conventional RR intervals combined to form a single RR n-interval (RRnI), N<<\hat{N} where \hat{N} is a total number of RRIs in the ECG data;
1≤n≤N;
x is an absolute variation multiple; and
pNNxn is a number of times an absolute difference between successive
RRₙIs exceeds xn milliseconds, expressed as a proportion of \hat{N}.

27. The system of claim 25, wherein x is 50.

28. The system of claim 22, wherein the predictive model is a machine learning model trained on past data from a pool of patients, to identify patterns in the values corresponding to the sepsis risk categories.

29. The system of claim 22, wherein the HRV parameters and/or HRnV parameters comprise at least one of a time domain parameter, a frequency domain parameter, a Poincare parameter, a deviation parameter, an entropy parameter, and a detrended fluctuation analysis (DFA) parameter.

30. The system of claim 22, wherein the HRV parameters and/or HRnV parameters comprise one or more parameters from Table 1.

31. A method for triaging sepsis patients, comprising:
receiving electrocardiogram data (ECG data) comprising data of a plurality of heart beats from a patient;
determining a value for each of a plurality of heart rate variability (HRV) metrics and each of a plurality of heart rate n-variability (HRnV) metrics from the ECG data;
transforming the heart rate n-variability metrics into respective vector representations;
determining one or more kernel metrics based on a combination of at least two vector representations;
applying a predictive model to the values and the kernel metrics to determine a sepsis risk category for the patient from a plurality of sepsis risk categories.

32. The method of claim 31, wherein the kernel metrics are determined using any one of: cosine similarity function, polynomial kernel function, sigmoid kernel function, RBF kernel function, Laplacian kernel function or Chi-squared kernel function.

33. The method of claim 31, further comprising receiving data, the data comprising at least one of patient demographic data and clinical data, wherein applying the predictive model to the values to determine a sepsis risk category for the patient comprises applying the predictive model to the patient demographic data.

34. The method of claim 31, wherein at least one of the HRnV parameters is NNxn, where:
N is a number of conventional RR intervals combined to form a single RR n-interval (RRₙI), N<<\hat{N} where \hat{N} is a total number of RRIs in the ECG data;
1≤n≤N;
x is an absolute variation multiple; and
NNxn is a number of times an absolute difference between successive RRnIs exceeds xn milliseconds.

35. The method of claim 31, wherein at least one of the HRₙV parameters is pNNxn, where:
N is a number of conventional RR intervals combined to form a single RR n-interval (RRnI), N<<\hat{N} where \hat{N} is a total number of RRIs in the ECG data;
1≤n≤N;
x is an absolute variation multiple; and
pNNxn is a number of times an absolute difference between successive RRnIs exceeds xn milliseconds, expressed as a proportion of \hat{N}.

36. The method of claim 34, wherein x is 50.

37. The method of claim 31, wherein the predictive model is a machine learning model trained on past data from a pool of patients, to identify patterns in the values corresponding to the sepsis risk categories.

38. The method of claim 31, wherein determining a value of each of a plurality of HRV parameters and each of a plurality of HRnV parameters comprises determining at least one of a time domain parameter, a frequency domain parameter, a Poincare parameter, a deviation parameter, an entropy parameter, and a detrended fluctuation analysis (DFA) parameter.

39. The method of claim 31, wherein determining a value of each of a plurality of HRV parameters and each of a plurality of HRnV parameters comprises determining one or more parameters from Table 1.
